(19) United States
(12) Patent Application Publication
(10) Pub. No.: US 2001/0043912 A1
(43) Pub. Date: Nov. 22, 2001

(54) Hair care compositions containing selected frizz control agents

(76) Inventor: Daniel Wayne Michael, Maineville, OH (US)

Correspondence Address: Armina E. Matthews, The Procter & Gamble Company, Sharon Woods Technical Center, 11511 Reed Hartman Highway, Cincinnati, OH, 45241, US

(21) Appl. No.: 09/837,237

(22) Filed: Apr. 18, 2001

Related U.S. Application Data
(63) Continuation-in-part of application No. 09/541,685, filed on Apr. 3, 2000, now abandoned.

(51) Int. Cl. A61K 7/06

(52) U.S. Cl. 424/70.12

(57) ABSTRACT

Disclosed are hair care compositions containing selected frizz control agents and to methods of using said compositions for controlling hair frizz. The compositions a non-volatile polysiloxane resin, a frizz control active selected from dimethicone copolyols, PEG-modified glycerides, PEG-modified glyceryl fatty acid esters, and mixtures thereof, and a vehicle. The composition provides improved frizz control and improved hair feel from a hair care composition.

### CROSS REFERENCE TO RELATED APPLICATION

This application is a continuation-in-part of prior application Ser. No. 09/541,685 filed on Apr. 3, 2000.

### FIELD OF THE INVENTION

The present invention relates to hair care compositions containing selected frizz control agents and to methods of using said compositions for controlling hair frizz.

### BACKGROUND OF INVENTION

Hair is often subjected to a wide variety of insults that can cause damage. These include shampooing, rinsing, drying, heating, combing, styling, perming, coloring, exposure to the elements, etc. Thus, the hair is often in a dry, rough, lusterless or frizzy condition due to abrasion of the hair surface and removal of the hair's natural oils and other natural conditioning and moisturizing components. Additionally, hair is subjected to weather-related changes, such as changes in humidity, which can leave hair in a frizzy condition.

A variety of approaches have been developed to alleviate hair frizz. These include reactive chemistry approaches aimed at a permanent restructuring of hair, and application of oily leave-on products to weigh down hair. The use of reactive chemistry to permanently restructure hair is described, for example, in U.S. Pat. No. 5,520,909. U.S. Pat. No. 5,520,909 specifically describes the use of marcaptan hair-reducing reactions for relaxing curly or frizzy hair. U.S. Pat. No. 5,609,860 describes the use of keratin-reducing substances for relaxing curly or frizzy hair. Additionally, U.S. Pat. No. 5,419,895 describes the reduction of frizzy hair using a thiol compound and an agent for reducing skin irritation. The use of reactive chemistry provides a permanent frizz reduction benefit. However, the reactive chemistry methods and compositions are extremely harsh on the hair structure and can cause hair to split or break and can also result in a loss of hair shine. Skin and/or eye irritation from the relatively harsh chemicals used in reactive chemistry methods is also common.

Typically, leave-on conditioner type hair formulations provide advantages over other more permanent frizz reduction approaches. For example, leave-on formulations are typically less damaging to the hair. Also, leave-on formulations are more convenient because the consumer can use the product at any time and then wash the product out of the hair with one washing. Another benefit is that the product may be applied to parts of the hair most in need of the frizz control benefits.

Commonly, hair conditioning benefits are provided through the use of hair conditioning agents such as cationic surfactants, cationic polymers, silicone conditioning agents, hydrocarbon and other organic oils and solid aliphatics such as fatty alcohols. These conditioning agents are well known in the art. See, for example, WO-A-97/35542, WO-A-97/35545, WO-A-97/35546, all of which describe the use of conditioning agents in shampoo compositions. However, the conditioning agents known in the art are often impractical for using in the large amounts necessary to reduce hair frizz. Usage of large amounts of conditioning agents that work to control hair frizz by coating and weighing down the hair commonly results in a poor perception of hair cleanliness and hair feel.

The broad class of dimethicone copolyols is generally known in the art for conditioning benefits. See, for example, WO-A-99/17719, and U.S. Pat. No. 5,482,703, both of which describe the use of dimethicone copolyols for use in hair conditioning compositions.

Furthermore, it has recently been suggested that polysiloxane resins could be used as hair conditioning agents. For example, U.S. Pat. Nos. 5,684,112 and 5,817,302 to Berthiaume, et al., incorporated by reference herein, describe low viscosity organofunctionalised siloxysilicates and gives examples of their use in hair care compositions. However, this reference does not address the problem of providing frizz control hair care products that reduce frizz while retaining shine and conditioning benefits. Specifically, these known compositions are not easy to work through the hair and often cause the hair to feel excessively tacky or greasy.

Surprisingly, it has now been found that hair care compositions comprising selected dimethicone copolyol frizz control agents, and/or selected PEG modified frizz control agents, in combination with a polysiloxane resin, a lipid vehicle material, and a cationic surfactant vehicle material have increased efficacy for frizz control while retaining good conditioning/shine benefits and good hair feel and appearance.

### SUMMARY OF THE INVENTION

The present invention provides hair care compositions comprising:

a) from about 0.001% to about 5%, by weight of the composition, of a non-volatile polysiloxane resin;

b) from about 1% to about 50%, by weight of the composition, of a frizz control active selected from the group consisting of:

i) dimethicone copolyols having the structure:

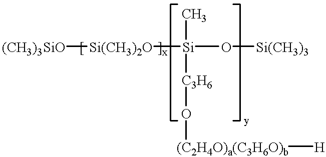

wherein x is equal to from about 3 to about 30, y is equal to from about 1 to about 10, a is equal to from 0 to about 100, and b is equal to from 0 to about 100, wherein at least one of either a or b is greater than 0 and wherein the dimethicone copolyol has an HLB value of about 14 or less,

ii) dimethicone copolyols having the structure:

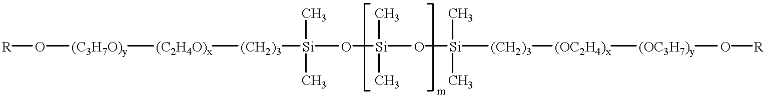

wherein R is independently selected from the group consisting of hydrogen, methyl, and combinations thereof, x is equal to from about 3 to about 30, y is equal to from about 1 to about 10, a is equal to from 0 to about 100, and b is equal to from 0 to about 100, wherein at least one of either a or b is greater than 0, and wherein the dimethicone copolyol has an HLB value of about 14 or less,

iii) PEG-modified glycerides having the structure:

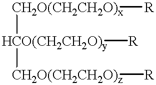

wherein one or more of the R groups is selected from saturated or unsaturated fatty acid moieties derived from animal or vegetable oils such as palmitic acid, lauric acid, oleic acid or linoleic acid wherein the fatty acid moieties have a carbon length chain of from 12 and 22, and wherein any other R groups are hydrogen, wherein x, y, and z, are individually equal to from 0 to about 45 and the average sum of x+y+z (the degree of ethoxylation) is equal to from about 10 to about 45, and mixtures thereof,

iv) PEG-modified glyceryl fatty acid esters having the structure:

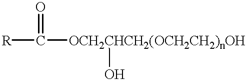

wherein R is an aliphatic group having from 12 to 22 carbon chain length and wherein n has an average value of from 5 to 40, and

v) mixtures thereof; and

c) from about 1.1% to about 13% of a vehicle which comprises;

i) a lipid material; and

ii) a cationic surfactant material.

All percentages herein are by weight of the total composition, unless otherwise indicated.

### DETAILED DESCRIPTION OF THE INVENTION

The frizz control compositions of this invention contain three essential ingredients: a non-volatile polysiloxane resin, a frizz control active, and a vehicle. These compositions encompass any composition form intended for human use on hair, including, for example, mousses, tonics, creams, and balms. Such composition forms may be dispensed through devices such as a pump or bottle and/or applied directly to the hair with the hands or another implement such as a comb or brush. Depending upon the specific frizz control and/or conditioning benefits and product theology desired, specific essential components may be selected, and other optional ingredients may be incorporated, in forming the final hair care product. The balance of the products is made up of water, preferably distilled water.

Specifically, the hair care compositions of the present invention comprise:

### Non-Volatile Polysiloxane Resin

The compositions of the present invention comprise from about 0.001% to about 5%, preferably from about 0.005% to about 3%, more preferably from about 0.01% to about 2%, even more preferably from about 0.1% to about 1%, by weight of the composition, of the non-volatile polysiloxane resin.

Polysiloxane resins are highly crosslinked polymeric siloxane systems. The crosslinking is introduced through the incorporation of trifunctional and tetrafunctional silanes with monofunctional or difunctional, or both, silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetrafunctional siloxane monomer units (and hence, a sufficient level of crosslinking) such that they dry down to a rigid, or hard, film are considered to be silicone resins. The ratio of oxygen atoms to silicon atoms is indicative of the level of crosslinking in a particular silicone material. Silicone materials, which have at least about 1.1 oxygen atoms per silicon atom will generally be silicone resins herein. Preferably, the ratio of oxygen atoms to silicon atoms is at least about 1.2:1.0. Silanes used in the manufacture of silicone resins include monomethyl, dimethyl, trimethyl, monophenyl, diphenyl, methylphenyl, ethylphenyl, propylphenyl, monovinyl, and methylvinylchlorosilanes, and tetrachlorosilane.

Silicone materials and silicone resins, in particular, can conveniently be identified according to a shorthand nomenclature system well known to those skilled in the art as “MDTQ” nomenclature. Under this system, the silicone is described according to presence of various siloxane monomer units which make up the silicone. Briefly, the symbol M denotes the monofunctional unit (CH₃)₃SiO_{0.5}; D denotes the difunctional unit (CH₃)₂SiO; T denotes the trifunctional unit (CH₃)SiO_{1.5}; and Q denotes the quadri- or tetra-functional unit SiO₂. Primes of the unit symbols, e.g., M′, D′, T′, and Q′ denote siloxane units with one or more substituents other than methyl, and must be specifically defined for each occurrence.

The polysiloxane resins for use herein preferably have at least one M′, D′, T′ or Q′ functionality that possesses a substituent group with delocalised electrons. The molar ratios of the various units, either in terms of subscripts to the symbols indicating the total number of each type of unit in the silicone (or an average thereof) or as specifically indicated ratios in combination with molecular weight complete the description of the silicone material under the MDTQ system.

Preferred polysiloxane resins for use herein are M′Q resins, more preferred are M′₆Q₃, M′₈Q₄ M′₁₀Q₅, M′₁₂Q₆ resins and mixtures thereof. Preferred M′Q resins are those which have at least one group containing delocalised electrons substituted on each M′ functionality. More preferred are resins where the other substituent groups are alkyl groups, especially preferred are methyl groups.

The polysiloxane resin for use herein preferably have at least one substituent group possessing delocalised electrons. This substituent or substituents can be independently selected from alkyl groups, aryl groups, alkoxy groups, alkaryl groups, arylalkyl arylalkoxy groups, alkaryloxy groups, and combinations thereof. Preferably, at least one of the resin substituent groups comprises an aryl group, arylalkyl group and/or alkaryl group. More preferably, at least one of the resin substituent groups comprises an alkaryl group and/or arylalkyl group substituent. More preferably, at least one of the resin substituent groups comprises an alkaryl group substituent. A particularly preferred alkaryl group substituent is 2-phenyl propyl.

Whereas at least one substituent preferably has delocalised electrons, the resins herein will also generally have other substituents without delocalised electrons. Such other substituents can include hydrogen, hydroxyl groups, alkyl groups, alkoxy groups, amino functionalities groups, and mixtures thereof. Preferred substituents without delocalised electrons are selected from alkyl group substituents, especially methyl group substituents. A particularly preferred methyl group substituent for use herein is dimethyl (2-phenylpropyl) silyl ester.

As used herein the term “aryl” means a functionality containing one or more homocyclic or heterocyclic rings. The aryl functionalities herein can be unsubstituted or substituted and generally contain from 3 to 16 carbon atoms. Preferred aryl groups include, but are not limited to, phenyl, naphthyl, cyclopentadienyl, anthracyl, pyrene, pyridine, pyrimidine

As used herein the term “alkyl” means a saturated or unsaturated, substituted or unsubstituted, straight or branched-chain, hydrocarbon having from 1 to 10 carbon atoms, preferably 1 to 4 carbon atoms. The term “alkyl” therefore includes alkenyls having from 2 to 8, preferably 2 to 4, carbons and alkenyls having from 2 to 8, preferably 2 to 4, carbons. Preferred alkyl groups include, but are not limited to, methyl, ethyl, propyl, isopropyl, and butyl. More preferred are methyl, ethyl and propyl.

As used herein the term “alkaryl” means a substituent comprising an alkyl moiety and an aryl moiety wherein the alkyl moiety is bonded to the siloxane resin.

As used herein the term “arylalkyl” means a substituent comprising an aryl moiety and an alkyl moiety wherein the aryl moiety is bonded to the siloxane resin.

The polysiloxane resins employed herein are non-volatile polysiloxane resins. The term “volatile” as used herein, unless otherwise specified, refers to those materials that are liquid under ambient conditions and have a vapor pressure as measured at 25 C. of at least about 0.01 mmHg, typically from about 0.01 mmHg to about 6.0 mmHg, Conversely, the term “nonvolatile” as used herein, unless otherwise specified, refers to those materials which are not volatile as that term is defined herein. Such “nonvolatile” materials will typically be in the form of a liquid, semi-solid or solid, and have no measurable vapor pressure as measured at 25 C.

The polysiloxane resins for use herein preferably have a viscosity of less than about 5000 mm²S⁻¹, more preferably less than about 2000 mm²S⁻¹, even more preferably less than about 1000 mm²s⁻¹, even more still preferably less than about 600 mm²s⁻¹, at 25° C. The viscosity can be measured by means of a Cannon-Fenske Routine Viscometer (ASTM D-445).

Background material on silicones including sections discussing silicone fluids, gums, and resins, as well as manufacture of silicones, can be found in Encyclopaedia of Polymer Science and Engineering (Volume 15, Second Edition, pp. 204-308, John Wiley & Sons, Inc., 1989), incorporated herein by reference. Background material on suitable polysiloxane resins including details of their manufacture can be found in U.S. Pat. Nos. 5,539,137; 5,672,338; 5,686,547 and 5,684,112, all of which are incorporated herein by reference.

### Frizz Control Active

The compositions of the present invention from about 1% to about 50% of a frizz control active. Preferably the compositions contain from about 2% to about 25%, more preferably from about 2% to about 10% of a frizz control active selected from:

i) dimethicone copolyols having the structure:

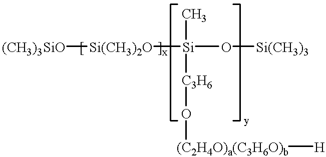

wherein x is equal to from about 3 to about 30, y is equal to from about 1 to about 10, a is equal to from 0 to about 100, and b is equal to from 0 to about 100, wherein at least one of either a or b is greater than 0 and wherein the HLB value is about 14 or less,

ii) linear type dimethicone copolyols having the structure:

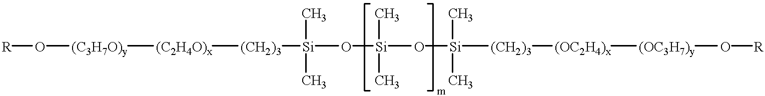

wherein the R group is hydrogen or methyl, wherein x is equal to from 0 to about 100, y is equal to from 0 to about 100, and m is equal to from about 1 to about 75, wherein at least one of either x or y is greater than 0, and wherein the HLB value is about 14 or less,

iii) PEG modified glycerides having the structure:

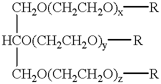

wherein one or more of the R groups is selected from saturated or unsaturated fatty acid moieties derived from animal or vegetable oils such as palmitic acid, lauric acid, oleic acid or linoleic acid wherein the fatty acid moieties have a carbon length chain of from 12 and 22, and wherein any other R groups are hydrogen, wherein x, y, and z, are individually equal to from 0 to about 45, and the average sum of x+y+z (the degree of ethoxylation) is equal to from about 10 to about 45,

iv) PEG modified glyceryl fatty acid esters corresponding to the formula:

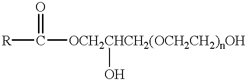

wherein R is an aliphatic group having from 12 to 22 carbon chain length, and wherein n (the degree of ethoxylation) is equal to from about 5 to about 40,

v) and mixtures thereof. Each of these classes of frizz control actives are described in detail as follows:

### a) Dimethicone Copolyol Frizz Control Actives

Dimethicone copolyols as a general class are well known in the art as containing conditioning agents. There are two subsets of dimethicone copolyols useful as frizz control actives in the present invention, those referred to in the art as the “comb” type and the “linear” type.

i) The first subset, the comb type, correspond to the formula:

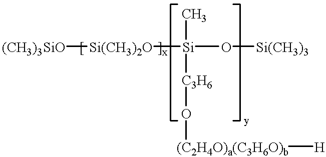

wherein x is equal to from about 3 to about 30, y is equal to from about 1 to about 10, a is equal to from 0 to about 100, and b is equal to from 0 to about 100, wherein at least one of either a or b is greater than 0 and the dimethicone copolyol has an HLB value of about 14 or less. Preferably the dimethicone copolyols have an HLB of about 12 or less and more preferably the dimethicone copolyols have an HLB of about 11 or less. Preferably the ratio of propylene oxide substituents (b) to ethylene oxide substituents (a) is at least about 2:1, more preferably at least about 3:1, even more preferably at least about 4:1, and most preferably the dimethicone copolyols have only propylene oxide substituents and no ethylene oxide substituents. Preferred commercially available comb type dimethicone copolyols, useful herein, include Abil B 8852®, and Abil B 8873® (manufactured by the Goldschmidt Chemical Corporation).

ii) The second subset, the linear type, correspond to the formula:

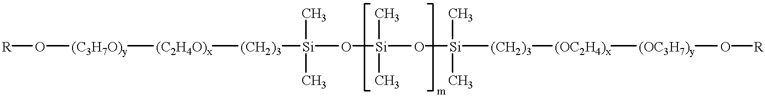

wherein R is independently selected from hydrogen and methyl groups, preferably hydrogen, wherein x is equal to from 0 to about 100, y is equal to from 0 to about 100, and m is equal to from about 1 to about 75, wherein at least one of either x or y is greater than 0, and the dimethicone copolyol has an HLB value of about 14 or less. Preferably the dimethicone copolyols have an HLB of about 12 or less and more preferably the dimethicone copolyols have an HLB of about 11 or less. Preferably the ratio of propylene oxide substituents (b) to ethylene oxide substituents (a) is at least about 2:1, more preferably at least about 3:1, even more preferably at least about 4:1, and most preferably the dimethicone copolyols have only propylene oxide substituents and no ethylene oxide substituents. A preferred commercially available linear type dimethicone copolyol, useful herein, is Abil B 8830® (manufactured by the Goldschmidt Chemical Corporation).

### b) PEG modified frizz control actives

iii) Also useful as frizz control actives herein are PEG-modified mono-, di- and triglyerides of the general formula:

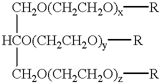

wherein one or more of the R groups is selected from saturated or unsaturated fatty acid moieties derived from animal or vegetable oils such as palmitic acid, lauric acid, oleic acid or linoleic acid wherein the fatty acid moieties have a carbon length chain of from 12 and 22, and wherein any other R groups are hydrogen, wherein x, y, and z, are individually equal to from 0 to about 45 and the average sum of x+y+z (the degree of ethoxylation) is equal to from about 10 to about 45, and mixtures thereof. Preferably the PEG-modified frizz control active has from 2 to 3 fatty acid R groups, more preferred are 3 fatty acid R groups (PEG-modified triglycerides). Preferably, the average sum of x+y+z (the degree of ethoxylation) is equal to from about 20 to 30, more preferred is an average sum of 25. Most preferred are PEG-substituted triglycerides having 3 oleic acid R groups, wherein the average degree of ethoxylation is about 25 (PEG-25 glyceryl trioleate).

Preferred commercially available PEG-modified triglycerides include Tagat TO®, Tegosoft GC, Tagat BL 276®, (all three manufactured by Goldschmidt Chemical Corporation) and Crovol A-40 (manufactured by Croda Corporation).

iv) Also useful herein are PEG-modified glyceryl fatty acid esters corresponding to the general formula:

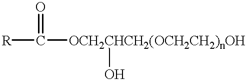

wherein R is an aliphatic group having from 12 to 22 carbon chain length and where n (the degree of ethoxylation) has an average value of from 5 to 40. Preferably, n has an average value of from about 15 to about 30, more preferred is an average value of from about 20 to about 30, and most preferably has an average value of 20. Preferred PEG-modified glyceryl fatty acid esters include PEG-30 glyceryl stearate and PEG-20 glyceryl stearate.

Preferred commercially available PEG-modified glyceryl fatty acid esters include Tagat S® and Tagat S 2® (manufactured by Goldschmidt Chemical Corporation).

### Vehicle

The present invention employs from about 1.1% to about 13%, preferably from about 1.1% to about 6%, more preferably from about 1.1% to about 3.5%, by weight of the composition, of a vehicle for the silicone conditioning agents. The vehicle, preferably a gel-type vehicle, comprises two essential components: a lipid material and a cationic surfactant material. Such gel-type vehicles are generally described in the following documents, both incorporated by reference herein: Barry, et al., “The Self-Bodying Action of Alkyltrimethylammonium Bromides/Cetostearyl Alcohol Mixed Emulsifiers; Influence of Quaternary Chain Length”, 35 J. of Colloid and Interface Science 689-708 (1971); and Barry, et al., “Rheology of Systems Containing Cetomacrogol 1000- Cetostearyl Alcohol, I. Self Bodying Action”, 38 J. of Colloid and Interface Science 616-625 (1972).

### Lipid Material

The vehicles of the present invention comprise one or more lipid materials, (herein referred to as comprising a “lipid material”, singly or in combination) which are essentially water-insoluble, and contain hydrophobic and hydrophilic moieties. The compositions of the present invention comprises from about 1% to about 10%, preferably from about 1% to about 5%, and more preferably from about 1% to about 3%, by weight of the composition, of the lipid material. Lipid materials useful herein include naturally or synthetically-derived acids, acid derivatives, alcohols, esters, ethers, ketones, and amides having carbon chains of from 12 to 22, preferably from 16 to 18, carbon atoms in length. Fatty alcohols and fatty esters are preferred; fatty alcohols are particularly preferred.

Lipid materials among those useful herein are disclosed in Bailey's Industrial Oil and Fat products, (3d edition, D. Swem, ed. 1979) (incorporated by reference herein ). Fatty alcohols included among those useful herein are disclosed in the following documents, all incorporated by reference herein: U.S. Pat. No. 3,155,591, Hilfer, issued Nov. 3, 1964; U.S. Pat. No. 4,165,369, Watanabe, et al., issued Aug. 21, 1979; U.S. Pat. No. 4,269,824, Villamarin, et al., issued May 26, 1981; British Specification 1,532,585, published Nov. 15, 1978; and Fukushima, et al., “The Effect of Cetostearyl Alcohol in Cosmetic Emulsions”, 98 Cosmetics & Toiletries 89-102 (1983). Fatty esters included among those useful herein are disclosed in U.S. Pat. No. 3,341,465, Kaufman, et al., issued Sep. 12, 1967 (incorporated by reference herein.)

Preferred esters for use herein include cetyl palmitate and glycerol monostearate. Cetyl alcohol and stearyl alcohol are preferred alcohols. A particularly preferred lipid material is comprised of a mixture of cetyl alcohol and stearyl alcohol containing from about 55% to about 65% (by weight of mixture) of cetyl alcohol and from about 35% to about 45% (by weight of mixture) of stearyl alcohol.

### Cationic Surfactant Material

The vehicle employed in the present invention also comprises one or more cationic surfactants, herein referred to as comprising (either singly or in combination) a “cationic surfactant material”. The compositions of the present invention comprise from about 0.1% to about 3%, preferably from about 0.1% to about 1%, more preferably from about 0.1% to about 0.5%, by weight of the composition, of the cationic surfactant material. Such cationic surfactants contain amino or quaternary ammonium hydrophilic moieties which are positively charged when dissolved in the aqueous composition of the present invention. Cationic surfactant vehicle materials among those useful herein are disclosed in the following documents, all incorporated by reference herein: M.C. Publishing Co., McCutcheon's, Detergents & Emulsifiers, (North American edition 1979); Schwartz, et al., Surface Active Agents, Their Chemistry and Technology, New York: Interscience Publishers, 1949; U.S. Pat. No. 3,155,591, Hilfer, issued Nov. 3, 1964; U.S. Pat. No. 3,929,678, Laughlin, et al., issued Dec. 30, 1975; U.S. Pat. No. 3,959,461, Bailey, et al., issued May 25, 1976; and U.S. Pat. No. 4,387,090, Bolich, Jr., issued Jun. 7, 1983.

Among the quaternary ammonium-containing cationic surfactant materials useful herein are those of the general formula:

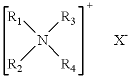

wherein R₁ is selected from hydrogen, an aliphatic group having from 1 to 22 carbon atoms, or an aromatic, aryl or alkylaryl group having from about 12 to about 22 carbon atoms; R₂ is an aliphatic group having from about 1 to about 22 carbon atoms; R₃ and R₄ are each alkyl groups having from about 1 to about 3 carbon atoms, and X is an anion selected from halogen, acetate, phosphate, nitrate and alkylsulfate radicals. The aliphatic groups may contain, in addition to carbon and hydrogen atoms, ether linkages, and other groups such as amido groups.

Other quaternary ammonium salts useful herein are of the formula:

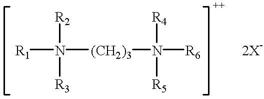

wherein R₁ is an aliphatic group having from 16 to 22 carbon atoms, R₂, R₃, R₄, R₅ and R₆ are independently selected from hydrogen and alkyl having from 1 to 4 carbon atoms, and X is an ion selected from halogen, acetate, phosphate, nitrate and alkyl sulfate radicals. Such quaternary ammonium salts include tallow propane diammonium dichloride.

Preferred quaternary ammonium salts include dialkyldimethylammonium chlorides, wherein the alkyl groups have from 12 to 22 carbon atoms and are derived from long-chain fatty acids, such as hydrogenated tallow fatty acid. (Tallow fatty acids give rise to quaternary compounds wherein R₁ and R₂ have predominately from 16 to 18 carbon atoms. ) Examples of quaternary ammonium salts useful in the present invention include ditallowdimethyl ammonium chloride, ditallowdimethyl ammonium methyl sulfate, dihexadecyl dimethyl ammonium chloride, di(hydrogenated tallow) dimethyl ammonium chloride, dioctadecyl dimethyl ammonium chloride, dieicosyl dimethyl ammonium chloride, didocosyl dimethyl ammonium chloride, di(hydrogenated tallow) dimethyl ammonium, acetate, dihexadecyl dimethyl ammonium chloride, dihexadecyl dimethyl ammonium acetate, ditallow dipropyl ammonium phosphate, ditallow dimethyl ammonium nitrate, di(coconutalkyl) dimethyl ammonium chloride, and stearyl dimethyl benzyl ammonium chloride. Ditallow dimethyl ammonium chloride, dicetyl dimethyl ammonium chloride, stearyl dimethyl benzyl ammonium chloride and cetyl trimethyl ammonium chloride are preferred quaternary ammonium salts useful herein. Di(hydrogenated tallow) dimethyl ammonium chloride is a particularly preferred quaternary ammonium salt.

Salts of primary, secondary and tertiary fatty amines are also preferred cationic surfactant vehicle materials. The alkyl groups of such amines preferably have from 12 to 22 carbon atoms, and may be substituted or unsubstituted. Secondary and tertiary amines are preferred, tertiary amines are particularly preferred. Such amines, useful herein, include stearamido propyl dimethyl amine, diethyl amino ethyl stearamide, dimethyl stearamine, dimethyl soyamine, soyamine, myristyl amine, tridecyl amine, ethyl stearylamine, N-tallowpropane diamine, ethoxylated (5 moles E.O.) stearylamine, dihydroxy ethyl stearylamine, and arachidylbehenylamine. Suitable amine salts include the halogen, acetate, phosphate, nitrate, citrate, lactate and alkyl sulfate salts. Such salts include stearylamine hydrochloride, soyamine chloride, stearylamine formate and N-tallowpropane diamine dichloride and stearamidopropyl dimethylamine citrate. Cationic amine surfactants included among those useful in the present invention are disclosed in U.S. Pat. No. 4,275,055, Nachtigal, et al., issued Jun. 23, 1981 (incorporated by reference herein.)

Preferred cationic surfactants for use herein are selected from cetyl trimethyl ammonium chloride, stearyl trimethyl ammonium chloride, tetradecyltrimethyl ammonium chloride, dicetyldimethyl ammonium chloride, dicocodimethyl ammonium chloride, and mixtures thereof.

### OPTIONAL COMPONENTS

### Cationic Conditioning Polymer

The hair compositions herein preferably employ a cationic hair conditioning polymer or mixtures of cationic hair conditioning polymers. If present, the cationic hair conditioning polymer is preferably employed at a level of from about 0.5% to about 10%, more preferably from about 2% to about 5%, even more preferably from about 1% to about 3% by weight of the composition.

The hair care compositions of the present invention may comprise one or more cationic polymers. As used herein, the term “polymer” includes materials whether made by polymerization of one type of monomer or made by two (i.e., copolymers) or more types of monomers.

Preferably, the cationic polymer is a water-soluble cationic polymer. As used herein, the term “water-soluble” cationic polymer, indicates a polymer which is sufficiently soluble in water to form a substantially clear solution to the naked eye at a concentration of 0.1% in water (distilled or equivalent) at 25° C. The preferred cationic polymer will be sufficiently soluble to form a substantially clear solution at 0.5% concentration, more preferably at 1.0% concentration.

The cationic polymers used herein will generally have a weight average molecular weight which is at least about 5,000, preferably from about 10,000 to about 10 million, more preferably, from about 100,000 to about 2 million. Most preferred are those cationic polymers having a weight average molecular weight of greater than about 900,000. The cationic polymer will generally have cationic nitrogen-containing moieties such as quaternary ammonium or cationic amino moieties, and mixtures thereof.

The cationic nitrogen-containing moiety or cationic amino moieties will be present generally as a substituent on a fraction of the total monomer units of the cationic hair conditioning polymers. Thus, the cationic polymer may comprise copolymers, terpolymers, etc. of quaternary ammonium or cationic amine-substituted monomer units and other non-cationic units referred to herein as spacer monomer units. Such polymers are known in the art, and a variety may be found in the CTFA Cosmetic Ingredient Dictionary, 3rd edition, edited by Estrin, Crosley, and Haynes, (The Cosmetic, Toiletry, and Fragrance Association, Inc., Washington, D.C., 1982).

The ‘cationic charge density” of a polymer refers to the ratio of the number of positive charges on a monomeric unit of which the polymer is comprises to the molecular weight of said monomeric unit. The cationic charge density of the cationic polymers useful herein are preferably at least about 0.1 meq/gram, more preferably at least about 0.5 meq/gram, even more preferably at least about 1.1 meq/gram, and still more preferably at least about 1.2 meq/gram, and most preferably at least about 1.5 meq/g. Generally, the cationic polymers will have a cationic charge density of less than about 5 meq/g, preferably less than 3.5 meq/g, more preferably less than about 2.5 meq/g and most preferably less than about 2.2 meq/g.

Cationic charge density of the cationic polymer may be determined according to the Kjeldahl Method. Those skilled in the art will recognize that the charge density of amino-containing polymers may vary depending upon pH and the isoelectric point of the amino groups. The charge density should be within the above limits at the pH of intended use.

Any anionic counterion may be utilized for the cationic polymers so long as the water solubility criteria is met. Suitable counterions include, for example, halides (e.g., Cl, Br, I, or F, preferably Cl, Br, or I), sulfate, and methylsulfate.

Suitable cationic polymers include, for example, copolymers of vinyl monomers having cationic amine or quaternary ammonium functionalities with water-soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone. The cationic amines may be primary, secondary, or tertiary amines, depending upon the particular species and the pH of the composition. In general, secondary and tertiary amines, especially tertiary amines, are preferred. The alkyl and dialkyl substituted monomers preferably have C1-C7 alkyl groups, more preferably C1-C3 alkyl groups. Other suitable spacer monomers include vinyl esters, vinyl alcohol (made by hydrolysis of polyvinyl acetate), maleic anhydride, propylene glycol, and ethylene glycol.

Amine-substituted vinyl monomers may be polymerized in the amine form, and then optionally may be converted to ammonium by a quaternization reaction. Amines may also be similarly quaternized subsequent to formation of the polymer. For example, tertiary amine functionalities may be quaternized by reaction with a salt of the formula R′X wherein R′ is a short chain alkyl, preferably a C1-C7 alkyl, more preferably a C1-C3 alkyl, and X− is an anion which forms a water-soluble salt with the quaternized ammonium.

Suitable cationic amino and quaternary ammonium monomers include, for example, vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts. The alkyl portions of these monomers are preferably lower alkyls such as the C1-C3 alkyls, more preferably C1 and C2 alkyls. Suitable amine-substituted vinyl monomers for use herein include dialkylaminoalkyl acrylate, dialkylaminoalkyl methacrylate, dialkylaminoalkyl acrylamide, and dialkylaminoalkyl methacrylamide, wherein the alkyl groups are preferably C1-C7 hydrocarbyls, more preferably C1-C3, alkyls.

The cationic polymers useful herein may comprise mixtures of monomer units derived from amine- and/or quaternary ammonium-substituted monomer and/or compatible spacer monomers.

Specific suitable cationic hair conditioning polymers include, for example: copolymers of 1-vinyl-2-pyrrolidone and 1-vinyl-3-methylimidazolium salt (e.g., chloride salt) (referred to in the industry by the Cosmetic, Toiletry, and Fragrance Association, “CTFA”, as Polyquaternium-16), such as those commercially available from BASF Wyandotte Corp. (Parsippany, N.J., USA) under the LUVIQUAT tradename (e.g., LUVIQUAT FC 370); copolymers of 1-vinyl-2-pyrrolidone and dimethylaminoethyl methacrylate (referred to in the industry by CTFA as Polyquaternium-11) such as those commercially available from Gaf Corporation (Wayne, N.J., USA) under the GAFQUAT tradename (e.g., GAFQUAT 755N); cationic diallyl quaternary ammonium-containing polymers, including, for example, dimethyldiallylammonium chloride homopolymer and copolymers of acrylamide and dimethyldiallylammonium chloride, referred to in the industry (CTFA) as Polyquaternium 6 and Polyquaternium 7, respectively; and mineral acid salts of amino-alkyl esters of homo- and co-polymers of unsaturated carboxylic acids having from 3 to 5 carbon atoms, as described in U.S. Pat. No. 4,009,256 issued to Nowack, et. al., on Feb. 22, 1977.

Preferred cationic polymers for use herein include cationic polysaccharide polymers, such as cationic cellulose derivatives and cationic starch derivatives.

Cationic polysaccharides useful in the present invention also include those polymers based on 5, 6 or 7 carbon sugars and derivatives which have been made water-soluble by, for example, derivatizing them with ethylene oxide. These polymers may be bonded via any of several arrangements, such as 1,4-α, 1,4-β, 1,3-α, 1,3-β and 1,6 linkages. The monomers may be in straight or branched chain geometric arrangements. Suitable examples include polymers based on arabinose monomers, polymers derived from xylose monomers, polymers derived from fucose monomers, polymers derived from fructose monomers, polymers based on acid-containing sugar monomers such as galacturonic acid and glucuronic acid, polymers based on amine sugar monomers such as galactosamine and glucosamine, particularly actylglucosamine, polymers based on 5 or 6 membered ring polyalcohol monomers, polymers based on gallactose monomers, polymers based on mannose monomers and polymers based on galcatomannan monomers.

Cationic polysaccharide polymer materials suitable for use herein include those of the formula:

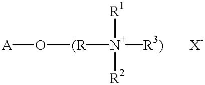

wherein: A is an anhydroglucose residual group, such as a starch or cellulose anhydroglucose residual, R is an alkylene oxyalkylene, polyoxyalkylene, or hydroxyalkylene group, or combination thereof, R1, R2, and R3 independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R1, R2 and R3) preferably being about 20 or less, and X⁻ is an anionic counterion, as previously described.

Cationic cellulose is available from Amerchol Corp. (Edison, N.J., USA) in their Polymer JR® and LR® series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200®. Another type of cationic cellulose includes the polymeric quaternary ammonium salts of hydroxyethyl cellulose reacted with diallyl dimethyl ammonium chloride, referred to in the industry (CTFA) as Polyquaternium 4, available from national Starch (Salisbury, N.C., USA).

Other cationic polymers that may be used include cationic guar gum derivatives, such as guar hydroxypropyltrimonium chloride (commercially available from Celanese Corp. in their Jaguar R series). Other materials include quaternary nitrogen-containing cellulose ethers (e.g., as described in U.S. Pat. No. 3,962,418, incorporated herein by reference), and copolymers of etherified cellulose and starch (e.g., as described in U.S. Pat. No. 3,958,581, incorporated herein by reference.)

As discussed above, the cationic polymer preferred for use herein is water soluble. This does not mean, however, that it must be soluble in the composition. Preferably the cationic polymer is either soluble in the composition, or in a complex coacervate phase in the composition formed by the cationic polymer and anionic material. Complex coacervates of the cationic polymer can be formed with anionic surfactants or with anionic polymers that can optionally be added to the compositions herein. An example of a useful anionic polymer is sodium polystyrene sulfonate.

### Thickening Agents

The compositions of the present invention may also comprise from about 0.02% to about 1%, preferably from about 0.05% to about 0.8%, more preferably from about 0.1% to about 0.5%, by weight of the composition, of a thickening agent selected from one or more associative polymers.

In the compositions according to the present invention, preferred associative polymers are nonionic associative polymers having an average molecular weight in the range of from about 2,000 to about 2,000,000, preferably from about 10,000 to about 1,000,000, more preferably from about 20,000 to about 800,000.

Associative polymers are a subclass of water-soluble polymers and are generally water-soluble macromolecular structures having both hydrophilic and hydrophobic components. Associative polymers can thicken compositions as a result of intermolecular association between the various water-insoluble hydrophobic components which form a part of, or are bonded to (directly or indirectly) a water-soluble polymer backbone (discussed in detail by G. D. Shay in Polymers in Aqueous Media, Advances in Chemistry series 223, pp. 467. Edited by J. E. Glass).

Associative polymers suitable for use in the compositions of the present invention include, but are not limited to, hydrophobically modified hydroxyalkyl cellulose polymers such as hydrophobically modified hydroxyethyl cellulose (HMHEC), hydrophobically modified alkoxylated urethane polymers, such as hydrophobically modified ethoxylated urethane (HEUR), and hydrophobically modified nonionic polyols. Preferred for use herein are hydrophobically modified hydroxyalkyl cellulose polymers and mixtures thereof. More preferred for use herein is cetyl hydroxyethyl cellulose.

### a) Hydrophobically Modified Hydroxyalkyl Cellulose Thickener

Cellulose ethers suitable for use herein, have, prior to hydrophobic modification, a sufficient degree of nonionic substitution selected from methyl, ethyl, hydroxyethyl and hydroxypropyl to cause them to be water-soluble. The preferred degree of nonionic substitution is in the range of from about 1.8 to about 4.0, preferably from about 2 to about 3, and especially from about 2.2 to about 2.8 by weight. The cellulose ethers are then further substituted with alkyl or alkenyl groups having from about 8 to about 30, preferably from about 10 to about 24, more preferably from about 14 to about 18 carbon atoms in an amount of from about 0.1 to about 1, preferably from about 0.3 to about 0.8, and especially from about 0.4 to about 0.6 weight percent. The cellulose ether to be modified is preferably one of low to medium molecular weight, i.e., less than 800,000 and preferably between 20,000 and 700,000 (75 to 2500 D.P.). Degree of polymerization (D.P.) as defined herein, means, the average number of glycoside units in the polymer.

Preferred cellulose ethers for use herein are selected from commercially available nonionic cellulose ethers such as hydroxyethylcellulose, hydroxy propylmethylcellulose, hydroxymethylcellulose, ethyl hydroxyethylcellulose and mixtures thereof.

The preferred cellulose ether substrate, for use herein, is a hydroxyethylcellulose (HEC) having a molecular weight ranging from about 50,000 to about 700,000. Hydroxyethylcellulose of this molecular weight is the most hydrophilic of the materials completed. Accordingly, control of the modification process and control of the properties of the modified product can be more precise with this substrate. Hydrophilicity of the most commonly used nonionic cellulose ethers varies in the general direction: hydroxyethyl>hydroxypropyl>hydroxypropyl methyl>methyl.

The long chain alkyl modifier, for the cellulose ether, can be attached to the cellulose ether substrate via an ether, ester or urethane linkage. The ether linkage is preferred. Although the modified cellulose ether materials are referred to as being “alkyl modified”, (the term alkyl as used generally herein also includes using alkenyl) it will be recognized that, except in the case where modification is effected with an alkyl halide, the modifier is not a simple long chain alkyl group. The group is actually an alphahydroxyalkyl radical in the case of an epoxide, a urethane radical in the case of an isocyanate, or an acyl radical in the case of an acid or acyl chloride. General methods for making modified cellulose ethers are taught in Landoll ('277) at column 2, lines 36-65.

Commercially available materials highly preferred for use herein include NATROSOL PLUS Grade 330 C (™), a hydrophobically modified hydroxyethylcellulose available from Aqualon Company, Wilmington, Del. This material has a C₁₆ alkyl substitution of from 0.4% to 0.8% by weight. The hydroxyethyl molar substitution for this material is from 3.0 to 3.7. The average molecular weight for the water-soluble cellulose prior to modification is approximately 300,000. Also suitable for use herein is NATROSOL PLUS Grade 430 CS (™)

Another material of this type is sold under the trade name NATROSOL PLUS CS Grade D-67 (™), by Aqualon Company, Wilmington, Del. This material has a C₁₆ substitution of from 0.50% to 0.95%, by weight. The hydroxyethyl molar substitution for this material is from 2.3 to 3.7. The average molecular weight for the water soluble cellulose prior to modification is approximately 700,000.

### Other Non-Essential Components

The hair care compositions of the present invention may also comprise a sensate. As used herein the term “sensate” means a substance that, when applied to the skin, causes a perceived sensation of a change in conditions, for example, but not limited to, heating, cooling, refreshing and the like. Sensates are preferably utilized at levels of from about 0.001% to about 10%, more preferably from about 0.005% to about 5%, even more preferably from about 0.01% to about 1%, by weight, of the total composition. Any sensate suitable for use in hair care compositions may be used herein. A non-limiting, exemplary list of suitable sensates can be found in GB-B-1315626, GB-B-1404596 and GB-B-1411785, all incorporated by reference herein. Preferred sensates for use in the compositions herein are camphor, menthol, 1-isopulegol, ethyl menthane carboxamide and trimethyl isopropyl butanamide.

The compositions of this invention may also contain optional components which may modify the physical and performance characteristics of the conditioning product. Such components include additional surfactants, salts, buffers, thickeners, solvents, opacifiers, pearlescent aids, preservatives, fragrance, colorants, dyes, pigments, chelators, sunscreens, vitamins, and medicinal agents. Optional components that are among those useful herein are disclosed in U.S. Pat. No. 4,387,090, Bolich, Jr., issued Jun. 7, 1983, incorporated by reference herein.

The frizz control compositions of the present invention may also optionally contain an anti-dandruff agent. The anti-dandruff agent provides the shampoo compositions with anti-microbial activity. The anti-dandruff agent may be particulate or soluble. Among the preferred type of anti-dandruff agents are particulate, crystalline anti-dandruff agents, such as sulfur, selenium sulfide and heavy metal salts of pyridinethione. Especially preferred is zinc pyridinethione. Soluble anti-dandruff agents, such as ketoconazole, are also known in the art. When present in the composition, the anti-dandruff agent comprises from about 0.1% to about 4%, by weight of the composition, preferably from about 0.1% to about 3%, most preferably from about 0.3% to about 2%, of the composition. Such anti-dandruff agent should be physically and chemically compatible with the essential components of the composition, and should not otherwise unduly impair product stability, aesthetics or performance.

The frizz control compositions of the present invention may also optionally contain one or more hair growth agents, such as zinc pyridinethione. The frizz control compositions of the present invention may also optionally contain a compound useful for regulating the growth and loss of hair. Such compounds known in the art include lupane triterpenes, derivatives of lupane triterpenes, derivatives of oleanane triterpenes, derivatives of ursane triterpenes, and salts and mixtures thereof, minoxidil (Rogaine®)(6-(1-piperidinyl)-2,4-pyrimidinediamine 3-oxide). See, U.S. Pat. Nos. 3,461,461; 3,973,061; 3,464,987; and 4,139,619. Another currently marketed product for promoting hair growth is Finasteride (Propecia®). See, EP 823436; U.S. Pat. No. 5,670,643; WO 97/15564; and WO 97/15558. Such hair growth/regulators should be physically and chemically compatible with the essential components of the composition and should not otherwise unduly impair product stability, aesthetics or performance.

The compositions of the present invention may contain additional surfactant materials, at levels such that the total level of surfactant present in the composition (including the cationic surfactant vehicle material, described above) is from about 0.01% to about 20%. These optional surfactant materials may be anionic, nonionic or amphoteric, such as ceteareth-20, steareth-20, sorbitan monoesters, sodium tallow alkylsulfate and tallow betaine. Optional surfactant materials are described in the following documents, all incorporated by reference herein: M. C. Publishing Co., McCutcheon's Detergents & Emulsifiers, (North American edition, 1979); Schwartz, et al., Surface Active Agents, Their Chemistry and Technology (1949); and U.S. Pat. No. 3,929,678, Laughlin, et al., issued Dec. 30, 1975.

Preferred optional surfactant materials, useful herein, are nonionic. Such surfactants are most commonly produced by the condensation of an alkylene oxide (hydrophilic in nature) with an organic hydrophobic compound, which is usually aliphatic or alkyl aromatic in nature. The length of the hydrophilic or polyalkylene moiety which is condensed with any particular hydrophobic compound can be readily adjusted to yield a water-soluble compound having the desired degree of balance between hydrophilic and hydrophobic elements. Such nonionic surfactants include polyethylene oxide condensates of alkyl phenols, condensation products of aliphatic alcohols with ethylene oxide, condensation products of ethylene oxide with a hydrophobic base formed by condensation of propylene oxide with propylene glycol, and condensation products of ethylene oxide with the product resulting from the reaction of propylene oxide and ethylene diamine. Another variety of nonionic surfactant is the non-polar nonionic, typified by the amine oxide surfactants. Preferred nonionic surfactants include ceteareth-20, steareth-20 and ceteth-2.

Salts and buffers may also be added in order to modify the product rheology. For example, salts such as potassium chloride and sodium chloride, may be added at levels of from about 0.001% to about 1%. Buffers, such as citrate or phosphate buffers, may also be used. Preferably the pH of the present compositions modified to a pH of from about 3 to about 10, more preferably from about 3 to about 7.5, even more preferably from about 6 to about 7.

Optional components may be incorporated which provide additional conditioning benefits. For example, proteins may be added at levels of from about 0.1% to about 10%. Cationic proteins may also serve as surfactant vehicle materials in the present invention.

### EXAMPLES

Compositions of the present invention are exemplified herein. These compositions can be prepared according to the following methodology:

1. Begin with charging 95-98% of the water into the formulation vessel. While agitating the water, any polysaccharide polymers that the formula contained are added. During the dissolution or dispersion of any polymers the mixture is heated to 30-35° C. When any added polymers are fully dispersed or dissolved, the mixture is heated to 80-85° C.

2. Once at this temperature, all materials comprising the vehicle and frizz control agents are added while mixing vigorously. While vigorous, the mixing is not sufficient to induce significant aeration. All other minor and optional ingredients are also added at this point, with the exception of the perfume and buffer materials, and/or any other materials whose character might be compromised by the 80-85 ° C. temperature. This mixing stage lasts from about 5-20 minutes to ensure thorough homogenization.

3. The mixture is then cooled at a rate controlled to protect overall product integrity (this can occur by any convenient means).

4. Once the temperature drops to 30 ° C., all additional ingredients are added and mixed for 10-30 minutes or until completely homogeneous. At this point, pH modifiers such as EDTA salts and/or Citric Acid salts are added to the composition to reach the desired pH level.

5. The product is then able to be removed from the mixing vessel and packed in any convenient manner.

EXAMPLES I-III - Frizz Control Creme

| | Weight % | | |
|---|---|---|---|
| | Example | Example | Example |
| Component | I | II | III |
| DRO Water | 90.94 | 86.94 | 90.94 |
| Polyquaterium-10¹ (Ucare KG30M) | 0.35 | 0.35 | 0.35 |
| Cetyl Alcohol | 1.20 | 1.20 | 1.20 |
| Stearyl Alcohol | 0.80 | 0.80 | 0.80 |
| PEG-60 Hydrogenated Castor Oil | 0.20 | 0.20 | 0.20 |
| Cetrimonium Chloride (CTMAC) | 0.49 | 0.49 | 0.49 |
| Dimethicone Copolyols (Abil 8830) | 4.00 | 4.00 | |
| PEG-25 Glycerol Trioleate | | 4.00 | 4.00 |
| (TAGAT TO) | | | |
| Minor Ingredients (perfumes, buffers, | 1.62 | 1.62 | 1.62 |
| etc.) | | | |
| Styryl Silicone | 0.20 | 0.20 | 0.20 |
| Cetyl Hydroxycellulose (Natrosol | 0.20 | 0.20 | 0.20 |
| 330C Plus) | | | |

EXAMPLES IV-VI - Frizz Control Creme

| | Weight % | | |
|---|---|---|---|
| | Example | Example | Example |
| Component | IV | V | VI |
| DRO Water | 90.74 | 86.74 | 90.74 |
| Polyquaterium-10¹ (Ucare KG30M) | 0.35 | 0.35 | 0.35 |
| Cetyl Alcohol | 1.20 | 1.20 | 1.20 |
| Stearyl Alcohol | 0.80 | 0.80 | 0.80 |
| PEG-60 Hydrogenated Castor Oil | 0.20 | 0.20 | 0.20 |
| Cetrimonium Chloride (CTMAC) | 0.49 | 0.49 | 0.49 |
| Dimethicone Copolyols (Abil 8830) | 4.00 | 4.00 | |
| PEG-25 Glycerol Trioleate | | 4.00 | 4.00 |
| (TAGAT TO) | | | |
| Citric Acid Anhydrous | 0.03 | 0.03 | 0.03 |
| Minor Ingredients (perfume, buffers, | 1.62 | 1.62 | 1.62 |
| etc.) | | | |
| Styryl Silicone | 0.20 | 0.20 | 0.20 |

EXAMPLES VII-IX - Frizz Control Composition

| | | | WEIGHT | WEIGHT | WEIGHT |
|---|---|---|---|---|---|
| Common Name | Trade Name | Manufacturer | % | % | % |
| Water | | | q.s. | q.s. | q.s. |
| Cetyl Alcohol | | | 3.0 | 3.00 | 3.0 |
| METHYLPARABEN | Methyl parasept | | 0.20 | 0.20 | 0.20 |
| PROPYLPARABEN | Nipasol M | | 0.10 | 0.10 | 0.10 |
| CTMAC | Dehyquart A | Henkel | 0.75 | 0.75 | 0.75 |
| Dimethicone Copolyol | Abil 8852 | Goldschmidt | 3.00 | | |
| PEG-7 Glyceryl Cocoate | Tegosoft GC | Goldschmidt | | 4.00 | |
| PEG-20 Almond | Crovol A-40 | Croda | | | 4.00 |
| Glycerides | | | | | |
| Citric Acid Anhydrous | same | | 0.025 | 0.025 | 0.025 |
| Na4EDTA | Hampene Na4 | | 0.11 | 0.11 | 0.11 |
| DMDM Hydantoin | Mackstat | | 0.20 | 0.20 | 0.20 |
| Cetyl Hydroxyethyl | Natrosol Plus | Aqualon | 0.2 | 0.1 | 0.1 |
| Cellulose | CS | | | | |
| Perfume Electric Youth | | | 0.50 | 0.50 | 0.50 |
| Styryl Silicone | Styryl MQ | GE | 0.20 | 0.20 | 0.20 |
| | Silicone | Silicones | | | |

EXAMPLES X-XII - Frizz Control Composition

| | | | WEIGHT | WEIGHT | WEIGHT |
|---|---|---|---|---|---|
| Common Name | Trade Name | Manufacturer | % | % | % |
| Water | | | q.s. | q.s. | q.s. |
| Cetyl Alcohol | | | 3.0 | 3.00 | 3.0 |
| METHYLPARABEN | Methyl parasept | | 0.20 | 0.20 | 0.20 |
| PROPYLPARABEN | Nipasol M | | 0.10 | 0.10 | 0.10 |
| CTMAC | Dehyquart A | Henkel | 0.75 | 0.75 | 0.75 |
| Dimethicone Copolyol | Abil 8852 | Goldschmidt | 3.00 | | |
| PEG-7 Glyceryl Cocoate | Tegosoft GC | Goldschmidt | | 4.00 | |
| PEG-20 Almond | Crovol A-40 | Croda | | | 4.00 |
| Glycerides | | | | | |
| Citric Acid Anhydrous | same | | 0.025 | 0.025 | 0.025 |
| Na4EDTA | Hampene Na4 | | 0.11 | 0.11 | 0.11 |
| DMDM Hydantoin | Mackstat | | 0.20 | 0.20 | 0.20 |
| Perfume | | | 0.50 | 0.50 | 0.50 |
| Styryl Silicone | Styryl MQ | GE | 0.20 | 0.20 | 0.20 |
| | Silicone | Silicones | | | |

What is claimed is:

1. A hair care composition comprising:
a) from about 0.001% to about 5%, by weight of the composition, of a non-volatile polysiloxane resin;
b) from about 1% to about 50%, by weight of the composition, of a frizz control active selected from the group consisting of:
i) dimethicone copolyols having the structure: 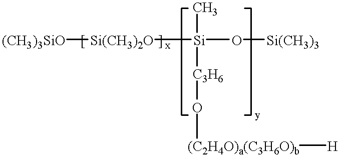
wherein x is equal to from about 3 to about 30, y is equal to from about 1 to about 10, a is equal to from 0 to about 100, and b is equal to from 0 to about 100, wherein at least one of either a or b is greater than 0 and wherein the dimethicone copolyol has an HLB value of about 14 or less,
ii) dimethicone copolyols having the structure: 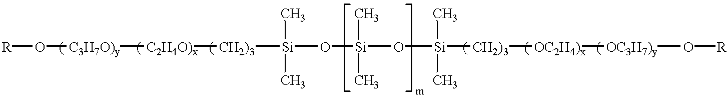
wherein R is selected from the group consisting of hydrogen, methyl, and combinations thereof, wherein x is equal to from 0 to about 100, y is equal to from 0 to about 100, and m is equal to from about 1 to about 75, wherein at least one of either x or y is greater than 0, and wherein the dimethicone copolyol has an HLB value of about 14 or less,
iii) PEG-modified glycerides having the structure: 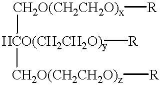
wherein one or more of the R groups is selected from saturated or unsaturated fatty acid moieties derived from animal or vegetable oils wherein the fatty acid moieties have a carbon length chain of from 12 and 22, and wherein any other R groups are hydrogen, wherein x, y, and z, are individually equal to from 0 to about 45 and the average sum of x+y+z is equal to from about 10 to about 45, and mixtures thereof,
iv) PEG-modified glyceryl fatty acid esters having the structure: 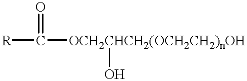
wherein R is an aliphatic group having from 12 to 22 carbon chain length and wherein n has an average value of from 5 to 40, and
v) mixtures thereof; and
c) from about 1.1% to about 13% of a vehicle which comprises;
i) a lipid material; and
ii) a cationic surfactant material.

2. A hair care composition according to claim 1, wherein at least one substituent group of the polysiloxane resin has delocalised electrons.

3. A hair care composition according to claim 2, wherein the resin substituent group or groups possessing the delocalised electrons is independently selected from the group consisting of aryl groups, arylalkyl groups, alkaryl groups, and mixtures thereof.

4. A hair care composition according to claim 3 wherein at least one resin substituent group comprises an alkaryl group.

5. A hair care composition according to claim 2 wherein the polysiloxane resin has a viscosity of less than about 5000 mm²S⁻¹ at 25° C.

6. A hair care composition according to claim 1, wherein the lipid material is selected from the group consisting of cetyl alcohol, stearyl alcohol, cetyl palmitate, glycerol monostearate, and mixtures thereof.

7. A hair care composition according to claim 6, wherein the lipid material comprises from about 55% to about 65% of cetyl alcohol and from about 35% to about 45% of stearyl alcohol, by weight of the lipid material.

8. A hair care composition according to claim 1, wherein the cationic surfactant is selected from the group consisting of cetyl trimethyl ammonium chloride, stearyl trimethyl ammonium chloride, tetradecyltrimethyl ammonium chloride, dicetyldimethyl ammonium chloride, dicocodimethyl ammonium chloride, and mixtures thereof.

9. A hair care composition according to claim 1, wherein the frizz control active is selected from the group consisting of the dimethicone copolyols having an HLB value of about 12 or less, the PEG-modified triglycerides wherein the sum of x+y+z is equal to from about 20 to 30, and the PEG-modified glyceryl fatty acid esters having an average n value of from about 15 to about 30, and mixtures thereof.

10. A hair care composition according to claim 1, wherein the composition comprises from about 2% to about 10%, by weight of the composition, of the frizz control active.

11. A hair care composition according to claim 1, wherein the composition is a leave-on composition.

12. A hair care composition according to claim 1 further comprising a cationic conditioning polymer.

13. A hair care composition according to claim 12, wherein the cationic conditioning polymer is selected from the group consisting of cationic polymers of saccharides, cationic copolymers of saccharides and mixtures thereof.

14. A hair care composition according to claim 1, wherein the composition further comprises a hydrophobically modified hydroxyalkyl cellulose thickener.

15. A hair care composition comprising:
a) a polysiloxane resin, wherein at least one substituent group of the resin has delocalised electrons;
b) a frizz control agent selected from the group consisting of dimethicone copolyols having an HLB value of about 11 or less and having only propylene oxide substituents, PEG-25 glyceryl trioleate, and mixtures thereof;
c) a vehicle which comprises:
i) cetyl alcohol;
ii) stearyl alcohol; and
iii) a cationic surfactant;
d) a cationic conditioning compound selected from the group consisting of cationic polymers and copolymers having a charge density of greater than about 1.5 meq/g and a weight average molecular weight of greater than about 900,000, and mixtures thereof; and
e) a hydrophobically modified hydroxyalkyl cellulose thickener.

16. A method of controlling hair frizz by applying to the hair an effective amount of a composition according to claim 1.

17. A method of conditioning the hair by applying to the hair an effective amount of a composition according to claim 1.

18. A packaged product comprising a composition according to claim 1 and a suitable package for said composition wherein the package has instructions indicating that the composition is intended to be left on the hair.

19. A packaged product comprising a composition according to claim 1 and a suitable package for said composition wherein the package has instructions indicating that the composition is intended to be applied to the hair to reduce frizz.

20. A method according to claim 1, wherein said composition is worked throughout the hair with the hands or a hair care implement.
